(19) United States
(12) Patent Application Publication
(10) Pub. No.: US 2017/0100322 A1
(43) Pub. Date: Apr. 13, 2017

(54) HAIR DYE WITH CATIONIC POLYMER

(71) Applicant: Henkel AG & Co. KGaA, Duesseldorf (DE)

(72) Inventors: Sylvia Kerl, Hamburg (DE); Susanne Hagenow, Hamburg (DE)

(73) Assignee: Henkel AG & Co. KGaA, Duesseldorf (DE)

(21) Appl. No.: 15/281,786

(22) Filed: Sep. 30, 2016

(30) Foreign Application Priority Data
Oct. 13, 2015 (DE) 10 2015 219 754.9

(51) Int. Cl. A61K 8/81; A61Q 5/12; A61Q 5/10

(52) U.S. Cl. CPC A61K 8/8182; A61Q 5/10; A61Q 5/12; A61K 2800/4324; A61K 2800/88; A61K 2800/882

(57) ABSTRACT

The present invention relates to a cosmetic agent for dyeing keratinic fibers, in particular human hair, including at least one quaternized copolymer of vinylpyrrolidone and dimethylaminoethyl methacrylate, and at least one oxidation dye precursor and/or a direct dye, wherein the use of the at least one quaternized copolymer of vinylpyrrolidone and dimethylaminoethyl methacrylate leads to improved conditioning, in particular to an increased volume, of the keratinic fibers with a simultaneously very low color shift.

### FIELD OF THE INVENTION

The present invention generally relates to a cosmetic agent for color modification of keratinic fibers with a special cationic polymer. Further, the invention comprises a packaging unit (kit of parts) and a method for dyeing keratinic fibers with the cosmetic agent.

### BACKGROUND OF THE INVENTION

Human hair is treated in many ways today with hair cosmetic preparations. These include, for instance, cleansing the hair with shampoos, care and regeneration with rinses and treatments, and bleaching, coloring, and shaping the hair using dyes, tints, waving compositions, and styling preparations. In this regard, agents for modifying or nuancing the color of head hair play a prominent role. Apart from bleaching compositions that bring about an oxidative lightening of the hair by breaking down the natural hair dyes, the oxidative hair dyeing is also of major importance in the field of hair dyeing.

So-called oxidation dyes are used for permanent, intensive colors with suitable fastness properties. Such dyes customarily include oxidation dye precursors, so-called developer components and coupler components. The developer components form the actual dyes under the influence of oxidizing agents or atmospheric oxygen with one another or upon coupling with one or more coupler components. The oxidation dyes are in fact characterized by excellent, long-lasting coloring results. For natural-looking colors, however, customarily a mixture of a relatively large number of oxidation dye precursors (ODP) must be used; in many cases, direct dyes (DD) are used, furthermore, for providing nuances.

In order to stabilize the dye precursors during storage and to accelerate the reaction during the oxidative application, oxidative dyes mostly have an alkaline pH, which is adjusted with alkalizing agents, such as alkanolamines, ammonia, or inorganic bases.

The aforementioned oxidation dye precursors (ODP) and alkalizing agents are typically incorporated into a cosmetically suitable carrier such as, for example, a cream or a gel. The carrier assures a homogeneous distribution and a sufficient residence time of the dye on the hair.

Commercial oxidation dyes are generally formulated in product series, which comprise a standardized carrier, which can be combined with the nuance-specific ODP combination and alkalizing agents with as little limitation as possible.

Consumers may usually obtain an indication of the hair color achievable with a hair dye from the packaging of the hair dye and/or a color chart enclosed in the packaging. It is very important for the consumer in this case that the result of the dyeing matches as accurately as possible the color indicated by the manufacturer.

The result of the dyeing depends not only on the employed combination of the ODP and optionally DD, but is also influenced by the ingredients of the carrier. For example, the dyes formed during the color development under the effect of the oxidizing agent or employed directly can have a very different absorption on the fibers; the carrier ingredients can also have a different effect on each dye.

Hair dyes are therefore tested comprehensively and extensively in regard to the achievable color and a plurality of application properties before introduction on the market. These tests always take into account the interactions between ODP and carriers only for a specific carrier. The manufacturer frequently wishes to match a hair dye series selectively to the special requirements of specific consumer groups by adding suitable active or care substances to the standardized carrier. The addition of one or more care substances with a hair-repairing action, for example, would be advisable for consumers with greatly damaged hair; the addition of one or more active substances increasing hair fullness/volume would be advisable for consumers with fine hair, whereas addition of too much conditioning agent would weigh down the hair further.

The addition of such additives nevertheless can have the result that differences arise between the hair color achieved with the additive-free (standard) carrier and the additive-containing carrier. Such color differences are called a “color shift” in the context of the present invention. This color shift, also called dE or ΔE, can be readily determined colorimetrically with a colorimeter, with which the colors in the L*,a*,b* color space are measured, for example, with a colorimeter from the company Datacolor, Spectraflash 450 type.

The L*,a*,b* color space is understood to be the CIELAB color space. The “L” value in this case stands for the lightness of the color (black-white axis); the higher the “L” value, the lighter the color. The “a” value stands for the red-green axis of the system; the higher this value, the more the color is shifted into red. The “b” value stands for the yellow-blue axis of the system; the higher this value, the greater the color is shifted into yellow.

The color shift ΔE, therefore the color difference between two (hair) colors, for each of which an L*,a*,b* value combination was determined, is calculated according to the following formula:

ΔE=(ΔL²+Δa²+Δb²)^{0.5}.

The higher the value for ΔE, the more pronounced the color difference or “color shift.” Color differences with ΔE<1 are not perceptible by the human eye. Color differences with ΔE<2 are visible to the trained eye. Color differences with ΔE>2 are also visible to the untrained eye.

In the worst case, the addition of an additive to a dye carrier causes a color shift versus the additive-free carrier (standard) with ΔE>2, which is also visible to the untrained eye of the consumer. To avoid having to perform complicated tests with each additive change of the standard carrier with respect to the achievable hair color and optionally the fastness properties, it is therefore desirable to identify active and care substances for the hair, the addition of which causes no or at least only a minor color shift.

The object forming the basis for the present invention, therefore, was to provide cosmetic agents for color modification of keratinic fibers, which include one or more active hair-conditioning substances that cause no or only a minimal color shift.

It was now found surprisingly that the addition of at least one special cationic polymer in cosmetic agents for dyeing keratinic fibers, in particular human hair, leads to improved conditioning, in particular to an increased volume, simultaneously with a color shift of ΔE<2 not visible to the untrained eye.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic agent for color modification of keratinic fibers, comprising in a cosmetically acceptable carrier at least one compound, selected from the group consisting of oxidation dye precursors, direct dyes, and mixtures thereof; and a quaternized copolymer of vinylpyrrolidone and dimethylaminoethyl methacrylate.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the invention is a cosmetic agent for color modification of keratinic fibers, comprising in a cosmetically acceptable carrier
a) at least one compound, selected from the group consisting of oxidation dye precursors, direct dyes, and mixtures thereof, and
b) a quaternized copolymer of vinylpyrrolidone and dimethylaminoethyl methacrylate.

The term “keratinic fibers or keratin fibers as well” are understood to be pelts, wool, feathers, and human hair. It is particularly preferred in the context of the present invention if the cosmetic agents are used for dyeing human hair.

In addition, the terms “volume” or “body” in the context of the present invention are understood to be the structural strength of a fiber mass. A higher volume or greater body confers more fullness on the fibers.

The agents include a cosmetic carrier. Preferably, the cosmetic carrier is aqueous, alcoholic, or aqueous-alcoholic. For example, creams, emulsions, gels, or surfactant-containing foaming solutions such as, for example, shampoos, foam aerosols, or other preparations, suitable for use on hair, are used in the context of the present invention.

An aqueous carrier in the context of the invention includes at least 30% by weight, particularly at least 50% by weight of water, based in each case on the total weight of the cosmetic agent.

Aqueous-alcoholic carriers in the context of the present invention are to be understood as water-containing compositions, including a C₁-C₄ alcohol in a total amount of 3 to 90% by weight, based in each case on the total weight of the cosmetic agent, in particular ethanol or isopropanol.

The agents can include in addition further organic solvents such as, for example, methoxybutanol, ethyl diglycol, 1,2-propylene glycol, n-propanol, n-butanol, n-butylene glycol, glycerol, diethylene glycol monoethyl ether, and diethylene glycol mono-n-butyl ether. Preferred in this case are all water-soluble organic solvents, the solvent being included in a total amount of 0.1 to 30% by weight, preferably of 1 to 20% by weight, and in particular of 2 to 10% by weight, based in each case on the total weight of the cosmetic agent.

The cosmetic agent includes as a first essential component a) a compound selected from the group of oxidation dye precursors (ODP), direct dyes (DD), and mixtures thereof.

In one preferred embodiment, the agents include at least one oxidation dye precursor.

Oxidation dye precursors based on their reaction behavior can be divided into two categories, so-called developer components and coupler components. Developer components can form the actual dye with themselves. They can therefore be present in the cosmetic agents as the only compounds. In one preferred embodiment, the cosmetic agents therefore include at least one oxidation dye precursor of the developer type. It can also be provided in the context of the present invention, however, that the cosmetic agents include at least one oxidation dye precursor of the coupler type. Especially good results are obtained in regard to dyeing keratinic fibers, if the cosmetic agents include at least one oxidation dye precursor of the developer type and at least one oxidation dye precursor of the coupler type.

The developer and coupler components are usually used in the free form. In the case of substances with amino groups, however, it can be preferred to use the salt form thereof, in particular in the form of the hydrochlorides and hydrobromides or sulfates.

Cosmetic agents are preferred that include the developer and/or coupler components each in a total amount of 0.001 to 10% by weight, primarily of 0.01 to 8% by weight, preferably of 0.1 to 5% by weight, and in particular of 0.5 to 3% by weight, based in each case on the total weight of the cosmetic agent.

Suitable oxidation dye precursors of the developer type are, for example, p-phenylenediamine and derivatives thereof. Preferred p-phenylenediamines are selected from one or more compounds of the group formed by p-phenylenediamine, p-toluylenediamine, 2-chloro-p-phenylenediamine, 2,3-dimethyl-p-phenylenediamine, 2,6-dimethyl-p-phenylenediamine, N,N-bis(2-hydroxyethyl)-p-phenylenediamine, 2-(2-hydroxyethyl)-p-phenylenediamine, 2-(1,2-dihydroxyethyl)-p-phenylenediamine, N-(2-hydroxypropyl)-p-phenylenediamine, N-(4′-aminophenyl)-p-phenylenediamine, 2-methoxymethyl-p-phenylenediamine, N-phenyl-p-phenylenediamine, 2-(2-hydroxyethyl)-p-phenylenediamine, and N-(4-amino-3-methylphenyl)-N-[3-(1H-imidazol-1-yl)propyl]amine, and the physiologically acceptable salts thereof.

It can be preferred, furthermore, to use as a developer component compounds that include at least two aromatic rings substituted with amino and/or hydroxyl groups. Preferred bicyclic developer components are selected from N,N′-bis(2-hydroxyethyl)-N,N′-bis(4′-aminophenyl)-1,3-diaminopropan-2-ol, N,N′-bis(4-aminophenyl)-1,4-diazacycloheptane, bis(2-hydroxy-5-aminophenyl)methane, and the physiologically acceptable salts thereof.

It can be preferred, furthermore, to use a p-aminophenol derivative or one of the physiologically acceptable salts thereof as a developer component. Preferred p-aminophenols are p-aminophenol, N-methyl-p-aminophenol, 4-amino-3-methylphenol, 4-amino-2-aminomethylphenol, 4-amino-2-(1,2-dihydroxyethyl)phenol, 4-amino-2-(diethylaminomethyl)phenol, and the physiologically acceptable salts thereof.

Further, the developer component can be selected from o-aminophenol and the derivatives thereof, preferably from 2-amino-4-methylphenol, 2-amino-5-methylphenol, 2-amino-4-chlorophenol, and/or the physiologically acceptable salts thereof.

Furthermore, the developer component can be selected from heterocyclic developer components, such as pyrimidine derivatives, pyrazole derivatives, pyrazolopyrimidine derivatives, or the physiologically acceptable salts thereof. Preferred pyrimidine derivatives are 2,4,5,6-tetraaminopyrimidine, 4-hydroxy-2,5,6-triaminopyrimidine, and the physiologically acceptable salts thereof. A preferred pyrazole derivative is 4,5-diamino-1-(2-hydroxyethyl)pyrazole and the physiologically acceptable salts thereof. Pyrazolo[1,5-a]pyrimidines are preferred in particular as pyrazolopyrimidines.

Preferred oxidation dye precursors of the developer type are selected from the group formed by p-phenylenediamine, p-toluylenediamine, 2-(2-hydroxyethyl)-p-phenylenediamine, 2-(1,2-dihydroxyethyl)-p-phenylenediamine, N,N-bis(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis(2-hydroxyethyl)-N,N′-bis(4-aminophenyl)-1,3-diaminopropan-2-ol, bis(2-hydroxy-5-aminophenyl)methane, 1,3-bis(2,5-diaminophenoxy)propan-2-ol, N,N′-bis(4-aminophenyl)-1,4-diazacycloheptane, 1,10-bis(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol and 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, or the physiologically acceptable salts of said compounds.

Particularly preferred developer components are p-toluylenediamine, 2-(2-hydroxyethyl)-p-phenylenediamine, 2-methoxymethyl-p-phenylenediamine, N-(4-amino-3-methylphenyl)-N-[3-(1H-imidazol-1-yl)propyl]amine, and/or 4,5-diamino-1-(2-hydroxyethyl)pyrazole, and the physiologically acceptable salts thereof.

According to a further preferred embodiment of the present invention, the cosmetic agent includes as the oxidation dye precursor, apart from at least one developer component, further in addition at least one coupler component. m-Phenylendiamine derivatives, naphthols, resorcinol and resorcinol derivatives, pyrazolones, and m-aminophenol derivatives are generally used as coupler components.

Preferred coupler components are selected from
(A)m-aminophenol and derivatives thereof, in particular 3-aminophenol, 5-amino-2-methylphenol, 3-amino-2-chloro-6-methylphenol, 5-amino-4-chloro-2-methylphenol, 5-(2′-hydroxyethyl)amino-2-methylphenol, and 2,4-dichloro-3-aminophenol,
(B) o-aminophenol and derivatives thereof, such as 2-amino-5-ethylphenol,
(C) m-diaminobenzene and derivatives thereof such as, for example, 2,4-diaminophenoxyethanol, 1,3-bis(2′,4′-diaminophenoxy)propane, 1-methoxy-2-amino-4-(2′-hydroxyethylamino)benzene, 2,6-bis(2′-hydroxyethylamino)-1-methylbenzene, 2-({3-[(2-hydroxyethyl)amino]-4-methoxy-5-methylphenyl}amino)ethanol, and 2-({3-[(2-hydroxyethyl)amino]-2-methoxy-5-methylphenyl}amino)ethanol,
(D) o-diaminobenzene and derivatives thereof,
(E) di- or trihydroxybenzene derivatives, in particular resorcinol, 2-chlororesorcinol, 4-chlororesorcinol, 2-methylresorcinol, and 1,2,4-trihydroxybenzene,
(F) pyridine derivatives, in particular 3-amino-2-methylamino-6-methoxypyridine, 2,6-diaminopyridine, 2,6-dihydroxy-3,4-dimethylpyridine, 2-amino-3-hydroxypyridine, and 3,5-diamino-2,6-dimethoxypyridine,
(G) naphthalene derivatives, such as 1-naphthol and 2-methyl-1-naphthol,
(H) morpholine derivatives, such as 6-hydroxybenzomorpholine,
(I) quinoxaline derivatives,
(J) pyrazole derivatives, such as 1-phenyl-3-methylpyrazol-5-one,
(K) indole derivatives, such as 6-hydroxyindole,
(L) pyrimidine derivatives, or
(M) methylenedioxybenzene derivatives, such as 1-(2′-hydroxyethyl)-amino-3,4-methylenedioxybenzene,
   and the physiologically acceptable salts thereof.

Preferred coupler components are selected from the group formed by 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy)ethanol, 1,3-bis(2,4-di aminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}-amino)ethanol, 2-[-morpholin-4-ylphenyl)amino]ethanol, 3-amino-4-(2-methoxyethoxy)-5-methylphenylamine, 1-amino-3-bis(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or the physiologically acceptable salts of the aforementioned compounds.

Particularly preferred coupler components are resorcinol, 2-methylresorcinol, 4-chlororesorcinol, 5-amino-2-methylphenol, 3-aminophenol, 2-(2,4-diaminophenoxy)ethanol, 1,3-bis(2,4-diaminophenoxy)propane, 1-methoxy-2-amino-4-(2′-hydroxyethylamino)benzene, 2-amino-3-hydroxypyridine, 2,6-dihydroxy-3,4-dimethylpyridine, 1,5-dihydroxynaphthalene, 2,7-dihydroxynaphthalene, and 1-naphthol, and the physiologically acceptable salts thereof.

In a particularly preferred embodiment of the present invention, the cosmetic agents are characterized in that they include as an oxidation dye precursor at least one developer component, selected from the group comprising p-phenylenediamine, p-toluylenediamine, N,N-bis(2-hydroxyethyl)amino-p-phenylenediamine, 1,3-bis[(2-hydroxyethyl-4′-aminophenyl)amino]propan-2-ol, 1,10-bis(2′,5′-diaminophenyl)-1,4,7,10-tetraoxadecane, 4-aminophenol, 4-amino-3-methylphenol, bis(5-amino-2-hydroxyphenyl)methane, 2,4,5,6-tetraaminopyrimidine, 2-hydroxy-4,5,6-triaminopyrimidine, 4,5-diamino-1-(2-hydroxyethyl)pyrazole, the physiologically acceptable salts thereof and mixtures thereof, and at least one coupler component, selected from the group comprising resorcinol, 2-methylresorcinol, 5-methylresorcinol, 2,5-dimethylresorcinol, 4-chlororesorcinol, resorcinol monomethyl ether, 5-aminophenol, 5-amino-2-methylphenol, 5-(2-hydroxyethyl)amino-2-methylphenol, 3-amino-4-chloro-2-methylphenol, 3-amino-2-chloro-6-methylphenol, 3-amino-2,4-dichlorophenol, 2,4-diaminophenoxyethanol, 2-amino-4-(2′-hydroxyethyl)aminoanisole sulfate, 1,3-bis(2,4-diaminophenoxy)propane, 2-amino-3-hydroxypyridine, 2-methylamino-3-amino-6-methoxypyri dine, 2,6-dihydroxy-3,4-dimethylpyridine, 3,5-diamino-2,6-dimethoxypyridine, 1-naphthol, 2-methyl-1-naphthol, 1,5-dihydroxynaphthalene, 2,7-dihydroxynaphthalene, 1-phenyl-3-methylpyrazol-5-one, 2,6-bis[(2′-hydroxyethyl)amino]toluene, 4-hydroxyindole, 6-hydroxyindole, 6-hydroxybenzomorpholine, the physiologically acceptable salts thereof, and mixtures thereof.

To obtain a balanced and subtle nuance formation, it can also be provided in the context of the present invention that the cosmetic agents with ODP in addition include at least one direct dye. Alternatively, also included are cosmetic agents in which the color modification originates only from direct dyes.

Direct dyes are dyes that are absorbed directly onto the hair and do not require any oxidative process to develop the color. Direct dyes are usually nitrophenylenediamines, nitroaminophenols, azo dyes, anthraquinones, or indophenols.

Direct dyes can be divided into anionic, cationic, and nonionic direct dyes.

Preferred anionic direct dyes are the compounds known under the names: Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, and Acid Black 52, as well as tetrabromophenol blue.

Preferred cationic direct dyes are cationic triphenylmethane dyes, such as Basic Blue 7, Basic Blue 26, Basic Violet 2, and Basic Violet 14, and aromatic systems, which are substituted with a quaternary nitrogen group, such as Basic Yellow 57, Basic Red 76, Basic Blue 99, Basic Brown 16, and Basic Brown 17, and HC Blue 16, as well as Basic Yellow 87, Basic Orange 31, and Basic Red 51.

Preferred nonionic direct dyes are HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, and 1,4-diamino-2-nitrobenzene, 2-amino-4-nitrophenol, 1,4-bis(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picraminic acid and salts thereof, 2-amino-6-chloro-4-nitrophenol, 4-ethylamino-3-nitrobenzoic acid, and 2-chloro-6-ethylamino-4-nitrophenol.

Furthermore, naturally occurring dyes as well can be used as direct dyes, as are found, for example, in henna red, henna neutral, henna black, chamomile blossoms, sandalwood, black tea, walnut, buckthorn bark, sage, logwood, madder root, catechu, and alkanna root.

The cosmetic agent preferably includes the direct dye in a total amount of 0.001 to 10% by weight, primarily of 0.01 to 8% by weight, preferably of 0.1 to 5% by weight, and in particular of 0.5 to 3% by weight, based in each case on the total weight of the cosmetic agent.

As a second essential component b), the cosmetic agents include at least one quaternized copolymer of vinylpyrrolidone and dimethylaminoethyl methacrylate.

The ratio of vinylpyrrolidone to dimethylaminoethyl methacrylate in the copolymer can vary over broad ranges. It can be preferred that the proportion of vinylpyrrolidone is higher than the proportion of dimethylaminoethyl methacrylate. It can be preferred in particular that the ratio of vinylpyrrolidone to dimethylaminoethyl methacrylate is in the range of 80:20.

The copolymer of vinylpyrrolidone and dimethylaminoethyl methacrylate is preferably quaternized with dimethyl sulfate and/or diethyl sulfate, diethyl sulfate being preferred. Copolymers of vinylpyrrolidone and dimethylaminoethyl methacrylate, quaternized with diethyl sulfate, are also known under the INCI name “Polyquaternium-11.”

The amount of the quaternized copolymer of vinylpyrrolidone and dimethylaminoethyl methacrylate in the cosmetic agent is preferably 0.1 to 10% by weight and more preferably 0.25 to 5% by weight, based in each case on the total weight of the cosmetic agent. High amounts of the quaternized copolymer of vinylpyrrolidone and dimethylaminoethyl methacrylate can lead to great, partially visible, color modifications, without further increasing the conditioning performance, in particular the volume of the treated keratinic fibers. An optimal ratio of the increase in volume to the color modification is obtained, if the amount of the quaternized copolymer of vinylpyrrolidone and dimethylaminoethyl methacrylate in the cosmetic agent is 0.5 to 1.5% by weight, based on the total weight of the cosmetic agent.

The cosmetic agents can include further active substances and additives. It is therefore preferred in the context of the present invention, if the cosmetic agent in addition includes at least one further compound, selected from the group comprising (i) thickeners; (ii) linear or branched, saturated or unsaturated alcohols having 8 to 20 carbon atoms; (iii) surfactants, in particular amphoteric surfactants; (iv) alkalizing agents; (v) oils; as well as (vi) mixtures thereof.

Preferably, the cosmetic agents are formulated as flowable preparations. In this case, the cosmetic agents should be formulated so that, on the one hand, they can be applied and distributed well at the application site but, on the other, are sufficiently viscous, so that they remain at the site of action during the contact time and do not run.

It has proven advantageous, therefore, if the cosmetic agents include at least one thickener from the group of (i) anionic, synthetic polymers; (ii) cationic, synthetic polymers; (iii) naturally occurring thickeners, such as nonionic guar gums, scleroglucan gums or xanthan gums, gum arabic, gum ghatti, karaya gum, tragacanth gum, carrageenan gum, agar-agar, locust bean flour, pectins, alginates, starch fractions and derivatives such as amylose, amylopectin, and dextrins, as well as cellulose derivatives such as, for example, methylcellulose, carboxyalkyl celluloses, and hydroxyalkyl celluloses; (iv) nonionic, synthetic polymers, such as polyvinyl alcohol or polyvinylpyrrolidinone; (v) inorganic thickeners, in particular phyllosilicates such as, for example, bentonite, particularly smectites, such as montmorillonite or hectorite; and (vi) mixtures thereof. The total amount of thickener is preferably 0.0005 to 5.0% by weight, more preferably 0.005 to 1.0% by weight, and particularly preferably 0.008 to 0.01% by weight, based in each case on the total weight of the cosmetic agent.

It can be preferred in the context of the present invention, if the cosmetic agent includes a linear or branched, saturated or unsaturated alcohol having 8 to 20 carbon atoms selected from the group comprising myristyl alcohol (1-tetradecanol), stearyl alcohol (1-octadecanol), cetearyl alcohol, 2-octyldodecanol, arachyl alcohol (eicosan-1-ol), gadoleyl alcohol ((9Z)-eicos-9-en-1-ol), arachidonyl alcohol ((5Z,8Z,11Z,14Z)-eicosa-5,8,11,14-tetraen-1-ol), preferably 2-octyldodecanol and/or cetearyl alcohol. The total amount of alcohol can be 1.0 to 35% by weight, preferably 5.0 to 30% by weight, preferably 10 to 25% by weight, and in particular 12 to 20% by weight, based in each case on the total weight of the cosmetic agent.

The cosmetic agents preferably furthermore can include at least one partial ester or polyol partial ester. Such partial esters are in particular the mono- and diesters of glycerol or the monoesters of propylene glycol or the mono- and diesters of ethylene glycol or the mono-, di-, tri-, and tetraesters of pentaerythritol in each case with linear saturated C₁₂-C₃₀ carboxylic acids, which may be hydroxylated, in particular those with palmitic and stearic acid, the sorbitan mono-, di-, or triesters of linear saturated C₁₂-C₃₀ carboxylic acids, which may be hydroxylated, in particular those of myristic acid, palmitic acid, stearic acid, or of mixtures of these fatty acids and the methyl glucose mono- and diesters of linear saturated C₁₂-C₃₀ carboxylic acids, which may be hydroxylated. The polyol partial esters are preferably selected from glycerol monostearate, glycerol monopalmitate, glycerol distearate, glycerol dipalmitate, ethylene glycol monostearate, ethylene glycol monopalmitate, ethylene glycol distearate, ethylen glycol dipalmitate, and mixtures thereof, in particular mixtures of glycerol monostearate, glycerol monopalmitate, glycerol distearate, and glycerol dipalmitate. The partial esters or polyol partial esters are each included in a total amount of 0.5 to 10% by weight, in particular of 3.0 to 8.0% by weight, based in each case on the total weight of the cosmetic agent.

The use of the alcohols, partial esters, and polyol partial esters listed above in the cosmetic agents can be particularly preferred when the cosmetic agents is present in the form of a cream-like oil-in-water emulsion.

It can be provided, furthermore, that the cosmetic agents include at least one surfactant.

According to one preferred embodiment of the present invention, the cosmetic agents include at least one amphoteric surfactant in a total amount of 0.1 to 5.0% by weight, in particular of 0.2 to 2.0% by weight, based in each case on the total weight of the cosmetic agent. Surface-active compounds that have at least one quaternary ammonium group and at least one —COO⁽⁻⁾ or —SO3⁽⁻⁾ group can be called amphoteric or zwitterionic surfactants.

The compounds listed below are particularly preferred as amphoteric surfactants in the content of the present invention:
alkyl betaines having 8 to 20 carbon atoms in the alkyl group,
amidopropyl betaines having 8 to 20 carbon atoms in the acyl group,
sulfobetaines having 8 to 20 carbon atoms in the acyl group, and
amphoacetates or amphodiacetates having 8 to 20 carbon atoms in the acyl group.

In a particularly preferred embodiment, the cosmetic agents include as a surfactant at least one amphoteric surfactant, selected from amidopropyl betaines having 9 to 13 carbon atoms in the acyl group, in a total amount of 0.1 to 5.0% by weight, in particular of 0.2 to 2.0% by weight, based in each case on the total weight of the cosmetic agent.

It can be provided, further, that the cosmetic agents include at least one ethoxylated nonionic surfactant. In this case, it has emerged as especially advantageous, if the ethoxylated nonionic surfactant has an HLB value above 10, preferably above 13. It is necessary to this end that the nonionic surfactant has a sufficiently high ethoxylation degree. In this regard, the cosmetic agent therefore includes as the ethoxylated nonionic surfactant at least one ethoxylated surfactant with at least 12 ethylene oxide units. Apart from the suitably ethoxylated fatty alcohols, in particular lauryl alcohol, myristyl alcohol, cetyl alcohol, stearyl alcohol, arachyl alcohol, and behenyl alcohol, in particular the adducts of 20 to 60 mol of ethylene oxide to castor oil and hydrogenated castor oil are especially suitable. The at least one ethoxylated nonionic surfactant is preferably selected from surfactants with the INCI name Ceteth-12, Steareth-12, Ceteareth-12, Ceteth-20, Steareth-20, Ceteareth-20, Ceteth-30, Steareth-30, Ceteareth-30, Oleth-30, Ceteareth-50, PEG-40 Hydrogenated Castor Oil, and PEG-60 Hydrogenated Castor Oil, and mixtures of these substances, particularly preferably selected from Ceteth-20, Steareth-20, Ceteareth-20, Ceteth-30, Steareth-30, and Ceteareth-30.

Cosmetic agents in the context of the present invention normally have a basic pH, in particular between pH 8.0 and pH 12. These pH values are necessary to assure an opening of the outer cuticle layer (cuticle) and to enable penetration of the oxidation dye precursors and/or the oxidizing agent into the hair.

The adjustment of the aforementioned pH can occur preferably with the use of an alkalizing agent. In the context of the present invention, the alkalizing agent is selected from the group of (i) inorganic alkalizing agents; (ii) organic alkalizing agents; and (iii) mixtures thereof, and is present in a total amount of 1.5 to 9.5% by weight, preferably of 2.5 to 8.5% by weight, preferably of 3.0 to 8.0% by weight, and in particular of 3.5 to 7.5% by weight, based in each case on the total weight of the cosmetic agent.

Preferred inorganic alkalizing agents are selected from the group formed by ammonia or ammonium hydroxide, therefore aqueous solutions of ammonia, sodium hydroxide, potassium hydroxide, calcium hydroxide, barium hydroxide, sodium phosphate, potassium phosphate, sodium silicate, potassium silicate, sodium carbonate, and potassium carbonate, and mixtures thereof. Ammonia or ammonium hydroxide is a particularly preferred alkalizing agent. Particularly preferred is ammonia in a total amount of 0.1 to 20% by weight, preferably of 0.5 to 10% by weight, in particular of 1.0 to 7.0% by weight, based in each case on the total weight of the cosmetic agent.

Preferred organic alkalizing agents are selected from at least one alkanolamine. Preferred alkanolamines are selected from alkanolamines from primary, secondary, or tertiary amines with a C₂-C₆ alkyl parent structure, bearing at least one hydroxyl group. Particularly preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol (monoisopropanolamine), 1-aminobutan-2-ol, 1-aminopentan-2-ol, 1-aminopentan-3-ol, 1-aminopentan-4-ol, 2-amino-2-methylpropanol, 2-amino-2-methylbutanol, 3-amino-2-methylpropan-1-ol, 1-amino-2-methylpropan-2-ol, 3-aminopropane-1,2-diol, 2-amino-2-methylpropane-1,3-diol, 2-amino-2-ethyl-1,3-propanediol, N,N-dimethylethanolamine, triethanolamine, diethanolamine, and triisopropanolamine. Very especially preferred alkanolamines are selected from the group comprising 2-aminoethan-1-ol (monoethanolamine), 2-amino-2-methylpropan-1-ol, 2-amino-2-methylpropane-1,3-diol, and triethanolamine. Particularly preferred cosmetic agents include a mixture of monoethanolamine and 2-amino-2-methylpropan-1-ol. Preferably the at least one alkanolamine is present in a total amount of 0.05 to 15% by weight, preferably of 0.5 to 10% by weight, and in particular of 3.5 to 7.5% by weight, based in each case on the total weight of the cosmetic agent.

Other preferred organic alkalizing agents are selected from basic amino acids, selected particularly preferably from the group formed by L-arginine, D-arginine, D/L-arginine, L-lysine, D-lysine, D/L-lysine, and mixtures thereof. Particularly preferred basic amino acids are selected from L-arginine, D-arginine, and D/L-arginine. Preferred cosmetic agents include at least one alkalizing agent, different from alkanolamines and ammonia, in a total amount of 0.05 to 5.0% by weight, in particular of 0.5 to 3.0% by weight, based in each case on the total weight of the cosmetic agent.

In a particularly preferred embodiment, the cosmetic agents include as alkalizing agents a mixture of at least two alkanolamines different from one another, in particular of monoethanolamine and 2-amino-2-methylpropan-1-ol, in a total amount of 0.05 to 15% by weight, preferably of 0.5 to 10% by weight, in particular of 3.5 to 7.5% by weight, based in each case on the total weight of the cosmetic agent.

Preferably, the pH of the cosmetic agents, measured at 22° C., is 8 to 12, primarily 9.5 to 12, preferably 10 to 11.5, and in particular 10.5 to 11.

In the context of the present invention, it can be preferred furthermore that the cosmetic agents include at least one oil, selected from the group comprising sunflower oil, corn oil, soy oil, pumpkin seed oil, grape seed oil, sesame oil, hazelnut oil, apricot kernel oil, macadamia nut oil, arara oil, castor oil, avocado oil, and mixtures thereof, in a total amount of 0.1 to 10% by weight, preferably of 0.2 to 5.0% by weight, and in particular of 0.5 to 2.0% by weight, based in each case on the total weight of the cosmetic agent. A care effect that does not have a negative effect on the volume of the keratinic fibers is achieved with the use of one of the aforesaid oils.

Particularly preferably, the cosmetic agents include grape seed oil in a total amount of 0.1 to 10% by weight, preferably of 0.2 to 5.0% by weight, and in particular of 0.5 to 2.0% by weight, based in each case on the total weight of the cosmetic agent.

According to a particularly preferred embodiment of the present invention, the cosmetic agents present as an oil-in-water emulsion contain, based in each case on the total weight of the cosmetic agents,
cetearyl alcohol in a total amount of 2.0 to 20% by weight, in particular of 5.0 to 12% by weight, further
mixtures of glycerol monostereate, glycerol monopalmitate, glycerol distearate, and glycerol dipalmitate in a total amount of 0.5 to 10% by weight, preferably 3.0 to 8.0% by weight, further
at least one amphoteric surfactant, selected from amidopropyl betaines having 9 to 13 carbon atoms in the acyl group, in a total amount of 0.1 to 5.0% by weight, in particular of 0.2 to 2.0% by weight, further
a mixture of at least two alkanolamines different from one another, in particular of monoethanolamine and 2-amino-2-methylpropan-1-ol, in a total amount of 0.05 to 15% by weight, preferably of 0.5 to 10% by weight, and in particular of 3.5 to 7.5% by weight, further
grape seed oil in a total amount of 0.1 to 10% by weight, preferably of 0.2 to 5.0% by weight, in particular of 0.5 to 2.0% by weight.

Oxidative dye compositions can also be prepared immediately before use from two or more separately packaged compositions. This lends itself in particular to separating incompatible ingredients in order to prevent a premature reaction. Separation into multi-component systems is preferred in particular when incompatibilities of the ingredients are a possibility or a risk. The oxidative dye composition in these cases is prepared by the consumer immediately before use by mixing the components. In the context of the present invention, this procedure in the case of oxidative dyes, in which the cosmetic agent is present initially separated from an oxidizing agent preparation including at least one oxidizing agent, is particularly preferred.

A further subject of the present invention therefore is a packaging unit (kit of parts), comprising, produced separately from one another,
a) at least one container (C1), including a cosmetic agent for color modification of keratinic fibers, and
b) at least one container (C2), including an oxidizing agent preparation, which in a cosmetically acceptable carrier includes at least one oxidizing agent in a total amount of 0.5 to 7.0% by weight, preferably of 1.0 to 7.0% by weight, and in particular of 3.0 to 7.0% by weight, based in each case on the total weight of the oxidizing agent preparation, and at least one acid.

The use of the at least one quaternized copolymer of vinylpyrrolidone and dimethylaminoethyl methacrylate in combination with specific amounts of oxidizing agents during use of the aforesaid packaging unit for dyeing keratinic fibers surprisingly results in increased conditioning, in particular in an increased volume, but without a color shift, perceptible by the untrained human eye, of ΔE>2 occurring due to the addition of the quaternized copolymer of vinylpyrrolidone and dimethylaminoethyl methacrylate.

The term “container” in the context of the present invention is understood to be an enclosure, which is present in the form of an optionally reclosable bottle, tube, a box, a small packet, a sachet, or similar enclosures. No limits are imposed on the enclosure material. Preferably, however, these are enclosures made of glass or plastic.

The oxidizing agents in the context of the present invention are different from atmospheric oxygen. Hydrogen peroxide and the solid adducts thereof to organic and inorganic compounds can be used as oxidizing agents. Suitable solid adducts are in particular the adducts to urea, melamine, polyvinylpyrrolidinone, and sodium borate. Hydrogen peroxide and/or one of its solid adducts to organic or inorganic compounds are particularly preferred as oxidizing agents. Preferably, the oxidizing agent is therefore selected from the group of persulfates, chlorites, hydrogen peroxide, and adducts of hydrogen peroxide to urea, melamine, and sodium borate, in particular hydrogen peroxide.

A particularly preferred embodiment of the present invention is therefore characterized in that hydrogen peroxide is present as the oxidizing agent in a total amount of 0.5 to 13% by weight, preferably of 1.0 to 13% by weight, and in particular of 3.0 to 13% by weight, based in each case on the total weight of the oxidizing agent preparation. The calculation of the total amount refers in this case to 100% H202.

The oxidizing agent preparations furthermore can include water in a total amount of 40 to 98% by weight, in particular of 65 to 85% by weight, based in each case on the total weight of the oxidizing agent preparation.

According to a particularly preferred embodiment of the present invention, the oxidizing agent preparations contain, based in each case on the total weight of the oxidizing agent preparations,
at least one linear saturated alkanol having 12 to 30 carbon atoms in a total amount of 0.1 to 10% by weight, preferably of 0.5 to 5.0% by weight, and in particular of 1.0 to 4.0% by weight, further
at least one ethoxylated nonionic surfactant, which is selected preferably from surfactants with the INCI name: Ceteth-12, Steareth-12, Ceteareth-12, Ceteth-20, Steareth-20, Ceteareth-20, Ceteth-30, Steareth-30, Ceteareth-30, Oleth-30, Ceteareth-50, PEG-40 Hydrogenated Castor Oil, and PEG-60 Hydrogenated Castor Oil, and mixtures of said substances, selected particularly preferably from Ceteth-20, Steareth-20, Ceteareth-20, Ceteth-30, Steareth-30, and Ceteareth-30, in a total amount of 0.1 to 10% by weight, preferably of 0.5 to 5.0% by weight, and in particular of 1.0 to 4.0% by weight, and
at least one ester from a carboxylic acid having 10 to 20 carbon atoms and a linear or branched alcohol having 1 to 5 carbon atoms, preferably isopropyl myristate, in a total amount of 3.0 to 25% by weight, preferably of 5.0 to 20% by weight, and in particular of 8.0 to 15% by weight.

The oxidizing agent preparations furthermore include at least one acid. Preferred acids are selected from dipicolinic acid, edible acids such as, for example, citric acid, acetic acid, malic acid, lactic acid, and tartaric acid, dilute mineral acids such as hydrochloric acid, phosphoric acid, pyrophosphoric acid, and sulfuric acid, and mixtures thereof. The oxidizing agent preparations preferably have a pH in the range of 2 to 5, in particular of 3 to 4.

To prepare oxidative dye compositions from the packaging unit (kit of parts), cosmetic agents, described above, for color modification of keratinic fibers in container C1 are mixed with the oxidizing agent preparation in container C2 or vice versa.

It can be especially advantageous, further, if the packaging unit includes at least one further hair treatment agent in an additional container, in particular a conditioning agent preparation. Said conditioning agent preparation advantageously includes at least one conditioning agent, selected from the group of further cationic polymers, silicone derivatives, and oils. Moreover, the packaging unit can comprise application aids, such as combs, brushes, dye brushes, or small brushes, personal protective clothing, in particular disposable gloves, and optionally instructions for use. A dye brush is understood to be a broad brush which has a point at the handle end which permits and simplifies the separation of fiber bundles or strands from the total amount of fibers.

The statements made about the cosmetic agents apply mutatis mutandis to the cosmetic agent in container C1 and the oxidizing agent preparation in container C2.

A further subject of the present invention is a method for dyeing keratinic fibers with increased conditioning with a simultaneously minimized color shift; the method comprises the following process steps:
a) providing a cosmetic agent for color modification of keratinic fibers (M1),
b) providing an oxidizing agent preparation (M2), including in a cosmetically acceptable carrier at least one oxidizing agent and at least one acid,
c) mixing the cosmetic agent (M1) with the oxidizing agent preparation (M2),
d) applying the mixture obtained in step c) to the keratinic fibers and leaving said mixture on the keratinic fibers for a time period of 1 to 60 minutes, preferably of 20 to 45 minutes, at room temperature and/or at at least 30° C.
e) rinsing the keratinic fibers with water and/or a cleaning composition, and
f) optionally applying an aftertreatment agent to the keratinic fibers and rinsing it off.

The above-described agent with a quaternized copolymer of vinylpyrrolidone and dimethylaminoethyl methacrylate is used as a cosmetic agent for color modification of keratinic fibers (M1).

The method for dyeing keratinic fibers with the use of a quaternized copolymer of vinylpyrrolidone and dimethylaminoethyl methacrylate results in improved conditioning, in particular an increased volume, of the dyed keratinic fibers, without, however, an undesirable color shift of ΔE>2, visible to the untrained human eye, occurring due to the addition of the quaternized copolymer of vinylpyrrolidone and dimethylaminoethyl methacrylate.

Room temperature in the context of the present invention is understood to be the ambient temperature. The effect of the coloring and/or lightening preparation can be intensified by an external heat supply, for example, by means of a heating hood. The preferred contact time of the coloring and/or lightening preparation on the keratinic fiber is 10 to 60 minutes, preferably 20 to 45 minutes. After the contact time ends, the remaining dye is washed out of the keratinic fibers with the aid of a cleaning preparation, which preferably includes at least one cationic and/or anionic and/or nonionic surfactant, and/or water. Optionally, the process is repeated with a further agent. After the washing out, the keratinic fibers are optionally rinsed with an aftertreatment agent, for example, a conditioning agent, and dried with a towel or a hot air dryer. The application of the dye preparation usually occurs by the hand of the user. Preferably, in this case, personal protective clothing is worn, in particular suitable protective gloves, for example, made of plastic or latex for a one-time use (disposable gloves), and optionally an apron. It is also possible, however, to apply the dye to the keratinic fibers with an application aid.

The statements made about the cosmetic agents and the packaging unit apply mutatis mutandis to the cosmetic agent M1, the oxidizing agent preparation M2, and further preferred embodiments of the method.

Lastly, a further subject of the present invention is the use of a cosmetic agent or a packaging unit (kit of parts) to increase the conditioning of keratinic fibers while simultaneously minimizing the color shift. The use of a quaternized copolymer of vinylpyrrolidone and dimethylaminoethyl methacrylate results in increased conditioning, in particular an increased volume, of the dyed keratinic fibers, without the addition of said active hair-conditioning substance leading to an undesirable color shift of ΔE>2, visible to the untrained human eye.

The statements made about the cosmetic agent and the packaging unit apply mutatis mutandis to further preferable embodiments of the use.

The following examples are intended to explain preferred embodiments of the invention but without limiting them.

### EXAMPLES

1. Formulations

Compositions of the employed cosmetic agents for color modification of keratinic fibers (oil-in-water emulsions, all amounts in % by weight).

| Raw material | V1 | E1 | V2 | E2 |
|---|---|---|---|---|
| Polyacrylic acid | 0.2 | 0.2 | 0.2 | 0.2 |
| Ammonia (25%) | 6 | 6 | 6 | 6 |
| 2-Octyldodecanol | 2 | 2 | 2 | 2 |
| C16-C18 fatty alcohol sulfate, Na | 0.8 | 0.8 | 0.8 | 0.8 |
| salt | | | | |
| Ethoxylated C12-C14 fatty | 4.5 | 4.5 | 4.5 | 4.5 |
| alcohol sulfate (2 EO), Na salt | | | | |
| KOH (50%) | 0.2 | 0.2 | 0.2 | 0.2 |
| Oleic acid | 0.4 | 0.4 | 0.4 | 0.4 |
| EDTA, tetra-Na salt | 0.2 | 0.2 | 0.2 | 0.2 |
| Glycerol monostearate | 4 | 4 | 4 | 4 |
| Cetearyl alcohol | 13 | 13 | 13 | 13 |
| Ethoxylated C12-C14 fatty | 3 | 3 | 3 | 3 |
| alcohol (20 EO) | | | | |
| Monoammonium phosphate | 0.001 | 0.001 | 0.001 | 0.001 |
| Titanium dioxide | 0.5 | 0.5 | 0.5 | 0.5 |
| Monoethanolamine | 0.3 | 0.3 | 0.3 | 0.3 |
| Sodium sulfite, anhydrous | 0.2 | 0.2 | 0.2 | 0.2 |
| Phospholipid | 0.1 | 0.1 | 0.1 | 0.1 |
| Polyquaternium-39 | 1.5 | 1.5 | 1.5 | 1.5 |
| Vitamin C | 0.1 | 0.1 | 0.1 | 0.1 |
| 2-Amino-2-methylpropanol | 0.1 | 0.1 | 0.1 | 0.1 |
| Perfume | 0.4 | 0.4 | 0.4 | 0.4 |
| 4-Amino-3-methylphenol | 0.2 | 0.2 | — | — |
| m-Aminophenol | 0.6 | 0.6 | 0.1 | 0.1 |
| p-Amino-o-cresol | 0.3 | 0.2 | — | — |
| Resorcinol | — | — | 0.2 | 0.2 |
| 4-Chlororesorcinol | — | — | 0.1 | 0.1 |
| 4,5-Diamino-1-(2- | 2 | 2 | — | — |
| hydroxyethyl)pyrazole•H₂SO₄ | | | | |
| p-Toluylenediamine sulfate | — | — | 1 | 1 |
| Copolymer of vinylpyrrolidone and | — | 1 | — | 1 |
| dimethylaminoethyl methacrylate | | | | |
| quaternized with diethyl sulfate | | | | |
| Water, demineralized | To 100 | To 100 | To 100 | To 100 |

The fat base was melted together at 80° C. and dispersed with a portion of the water amount. The remaining formulation components were then incorporated in sequence while stirring. The mixture was then made up with water to 100% by weight and the formulation stirred until cold.

Oxidizing agent preparation O1 (all amounts in % by weight)

| Raw material | O1 |
|---|---|
| Disodium pyrophosphate | 0.1 |
| Dipicolinic acid | 0.1 |
| Potassium hydroxide 50% | 0.3 |
| 1-Hydroxyethane-1,1-diphosphonic acid 60% | 0.4 |
| C₁₆₋₁₈ fatty alcohol sulfate | 0.3 |
| PEG-40 Castor Oil | 1 |
| Cetearyl alcohol | 4 |
| Ceteareth-20 | 1 |
| Beeswax | 0.3 |
| Isopropyl myristate | 10 |
| Hydrogen peroxide 50% | 11 |
| Water, demineralized | To 100 |

2. Color Shift Due to the Addition of the Quaternized Copolymer of Vinylpyrrolidone and Dimethylaminoethyl Methacrylate

To prepare the oxidative dye for determining the color shift, the cosmetic agents V1 and E1 were mixed in the weight ratio of 1:1 with the above oxidizing agent preparation O1.

The oxidative dyes prepared in this way were each applied in a defined amount (4 g of oxidative dye per 1 g of yak hair) to yak hair strands (12 strands each per oxidative dye) and remained on the hair strands for a contact time of 30 minutes at 32° C. Next, the remaining agents were each rinsed out of the hair strands for 2 minutes with lukewarm water; the strands were first dried with a towel and then blown dry.

All strands were measured with a colorimeter from the company Datacolor, Spectraflash 450 type. The ΔE values used for evaluating the color shift emerge from the L*a*b color values measured for each strand as follows:

ΔE=((Lᵢ−L₀)²+(aᵢ−a₀)²+(bᵢ−b₀)²)^{1/2}

a₀, b₀, and L₀ are hereby averages of measured color values determined from 12 measurements for the yak hair strands dyed during use of the standardized carrier. aᵢ, bᵢ, and Lᵢ stand for averages of measured color values, which were obtained for dyed yak hair strands with addition of the quaternized copolymer of vinylpyrrolidone and dimethylaminoethyl methacrylate to the standardized carrier.

The higher the value for ΔE, the more pronounced the color difference or “color shift.” Color differences with ΔE<1 are not perceptible by the human eye. Color differences with ΔE<2 are visible to the trained eye. Color differences with ΔE>2 are also visible to the untrained eye. The ΔE values for colors achieved with use of the cosmetic agent E1 are presented in Table 1. The color with the cosmetic agent E1, which includes the quaternized copolymer of vinylpyrrolidone and dimethylaminoethyl methacrylate in a total amount 1.0% by weight, has only a minor color shift of ΔE<1, which is not visible.

| Oxidative dye | ΔE |
|---|---|
| E1 + O1 (1:1) | 0.94 |

3. Increased Volume

To prepare the oxidative dye for determining the conditioning in the form of an increased volume, the cosmetic agents V2 and E2 were each mixed in the weight ratio of 1:1 with the above oxidizing agent preparation O1.

For the dyeing, 6 strands each of natural light-brown European hair 6/0 (Kerling International (Backnang), lot #09/2009, B3, length: 22 cm, weight: 2.8 g±0.05 g) were used per oxidative dye. To this end, 4 g in each case of the prepared oxidative dye was applied per 1 g of hair strands. After the strands were dyed for 30 minutes at 32° C., they were rinsed for 2 minutes with water and dried in air. The 12 dyed strands and the 6 reference strands that were not dyed were stored for at least 48 hours at 25° C. and 50% relative humidity.

The volume (body) was measured as follows:
Before the measurement, each strand was twisted using a machine. For this purpose, each strand was fixed at its bottom end and then the top end was rotated 5 times about its own axis. The twisted strands were blown dry for 45 minutes at 45° C. After this, the strands were stored for 16 hours in the twisted state at 23° C. and 50% relative humidity before they were loosened. Next, each strand was combed carefully 5 times with the coarse-toothed side and 5 times with the fine-toothed side of a comb (Hercules Sagemann). Immediately after the combing, each strand was fixed in a load cell and the top end of the strand was centered in a ring made of polished steel with an inner diameter of 10 mm. While the strands were pulled through the ring with a constant velocity of 100 mm/min, the required force and the distance traveled were measured.

The obtained measured values are compared using the software Statistica 12.0 (StatSoft Inc., USA).

| Strands | Force [N] |
|---|---|
| Reference | 0.019 |
| Dyed with V2 + O1 (1:1) | 0.021 |
| Dyed with E2 + O1 (1:1) | 0.025 |

The volume of the hair strands is the higher, the greater the maximum exerted force.

Doubling the amount of quaternized copolymer of vinylpyrrolidone and dimethylaminoethyl methacrylate in the cosmetic agent E2 bought about no further advantage in regard to volume but had a negative effect on hair feel.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent for color modification of keratinic fibers, comprising, in a cosmetically acceptable carrier:
a) at least one compound, selected from the group consisting of oxidation dye precursors, direct dyes, and mixtures thereof, and
b) a quaternized copolymer of vinylpyrrolidone and dimethylaminoethyl methacrylate.

2. The cosmetic agent according to claim 1, wherein the copolymer of vinylpyrrolidone and dimethylaminoethyl methacrylate is quaternized with dimethyl sulfate and/or diethyl sulfate.

3. The cosmetic agent according to claim 1, wherein the copolymer of vinylpyrrolidone and dimethylaminoethyl methacrylate is quaternized with diethyl sulfate.

4. The cosmetic agent according to claim 1, wherein the amount of quaternized copolymer of vinylpyrrolidone and dimethylaminoethyl methacrylate is 0.1 to 10% by weight based in each case on the total weight of the cosmetic agent.

5. The cosmetic agent according to claim 1, wherein the amount of quaternized copolymer of vinylpyrrolidone and dimethylaminoethyl methacrylate is 0.25 to 5% by weight based in each case on the total weight of the cosmetic agent.

6. The cosmetic agent according to claim 1, wherein the amount of quaternized copolymer of vinylpyrrolidone and dimethylaminoethyl methacrylate is 0.5 to 1.5% by weight based in each case on the total weight of the cosmetic agent.

7. The cosmetic agent according to claim 1, further comprising 1.0 to 35% by weight, based on the total weight of the cosmetic agent, of a linear or branched, saturated or unsaturated alcohol having 8 to 20 carbon atoms.

8. The cosmetic agent according to claim 1, further comprising 10 to 25% by weight, based on the total weight of the cosmetic agent, of a linear or branched, saturated or unsaturated alcohol having 8 to 20 carbon atoms.

9. The cosmetic agent according to claim 1, further comprising 12 to 20% by weight, based on the total weight of the cosmetic agent, of a linear or branched, saturated or unsaturated alcohol having 8 to 20 carbon atoms.

10. The cosmetic agent according to claim 7, wherein the linear or branched, saturated or unsaturated alcohol is 2-octyldodecanol and/or cetearyl alcohol.

11. The cosmetic agent according to claim 1, further comprising 0.5 to 10% by weight, based on the total weight of the cosmetic agent, of partial esters and/or polyol partial esters.

12. The cosmetic agent according to claim 1, further comprising 3.0 to 8.0% by weight, based on the total weight of the cosmetic agent, of partial esters and/or polyol partial esters.

13. A kit comprising, produced separately from one another,
a) at least one container (C1), including a cosmetic agent for color modification of keratinic fibers according to claim 1, and
b) at least one container (C2), including an oxidizing agent preparation, which in a cosmetically acceptable carrier includes at least one oxidizing agent in a total amount of 0.5 to 7.0% by weight based on the total weight of the oxidizing agent preparation, and at least one acid.

14. A method for dyeing keratinic fibers with increased conditioning, in particular with an increased volume, of the dyed keratinic fibers, with a simultaneously minimized color shift, the method comprising the following process steps:
a) providing a cosmetic agent for color modification of keratinic fibers (M1) according to one of claim 1,
b) providing at least one oxidizing agent preparation (M2), including in a cosmetically acceptable carrier at least one oxidizing agent in a total amount of 0.5 to 7.0% by weight, based on the total weight of the oxidizing agent preparation, and at least one acid,
c) mixing the cosmetic agent (M1) with the oxidizing agent preparation (M2),
d) applying the mixture obtained in step c) to the keratinic fibers and leaving said mixture on the keratinic fibers for a time period of 1 to 60 minutes at room temperature and/or at at least 30° C.
e) rinsing the keratinic fibers with water and/or a cleaning composition, and
f) optionally applying an aftertreatment agent to the keratinic fibers and rinsing it off.
